(19) United States
(12) Patent Application Publication
(10) Pub. No.: US 2007/0167967 A1
(43) Pub. Date: Jul. 19, 2007

(54) Medical procedure via natural orifice and puncture device

(75) Inventors: Takayasu Mikkaichi, Tokyo (JP); Takahiro Kogasaka, Tokyo (JP); Kensei Nakahashi, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., TOKYO (JP)

(21) Appl. No.: 11/331,975

(22) Filed: Jan. 13, 2006

(51) Int. Cl. A61B 17/34

(52) U.S. Cl. 606/185

(57) ABSTRACT

A medical procedure performed via a natural orifice comprises: inserting a puncture device from an abdominal wall side into the vicinity of a target site in a hollow organ, and protruding the distal end thereof inside the hollow organ; introducing an observation device into the hollow organ via a natural orifice of a living body, and setting a target site at the hollow organ by checking the puncture device as an indicator; and treating the target site confirmed by the observation device by using an treatment device inserted via a natural orifice.

### BACKGROUND OF THE INVENTION

1. Technical Field

The present invention relates to a medical procedure via a natural orifice of a living body and a puncture device used in medical procedures.

2. Description of the Related Art

When a medical procedure (including observation and treatment, the same shall apply hereinafter) are performed for human organs, such laparoscopic surgery is known that in place of widely dissecting an abdominal wall, a plurality of openings are formed on the abdominal wall to insert treatment devices, for example, a rigid laparoscope and forceps, into individual openings for procedures. Such surgery provides the benefit of less invasive because only small orifices need to be opened in the abdominal wall.

In recent years, methods of performing procedures upon inserting a flexible endoscope via a mouth, nose, anus, or other natural orifices of a patient have been proposed as methods of further reducing the burden on a patient. An example of such a procedure is disclosed in U.S. Pat. No. 5,458,131. With this method, a flexible endoscope is inserted from a mouth of a patient who has been treated by insufflation, an opening is formed in a stomach wall, and the endoscope is fed into an abdominal cavity from this opening. The endoscope is used for monitoring the abdominal cavity. Further, an organ is treated by using an treatment device inserted through the endoscope and an treatment device inserted through another opening formed in the stomach or inserted through an opening formed in the sigmoid colon from the anus. At the completion of procedures for the abdominal cavity, the treatment devices are removed to close the opening. In closing the opening, tissues around the opening are aspirated so as to be put together and clamped by using an O-ring.

### SUMMARY OF THE INVENTION

The present invention is to provide a method for confirming more easily a specific site of a hollow organ by using an observation device introduced via a natural orifice and a device therefor.

A medical procedure performed via a natural orifice according to a first aspect of the present invention comprises: inserting a puncture device from an abdominal wall side into the vicinity of a target site in a hollow organ, and protruding the distal end thereof inside the hollow organ; introducing an observation device into the hollow organ via a natural orifice of a living body, and setting a target site at the hollow organ by checking the puncture device as an indicator; and treating the target site confirmed by the observation device by using an treatment device inserted via a natural orifice.

A puncture device according to a second aspect of the present invention comprises: a needle configured to be inserted from an abdominal wall into a hollow organ; a first balloon attached to the needle and inflatable by supply of a fluid; a second balloon attached to a site closer to the distal end side of the needle than a site to which the first balloon is attached and inflatable by supply of the fluid; and a duct which supplies the fluid respectively to the first balloon and the second balloon.

### BRIEF DESCRIPTION OF THE DRAWINGS

FIG. 1A is a drawing showing a diathermy knife inserted into an endoscope used in the embodiment;

FIG. 1B is an enlarged view showing the distal end of the endoscope and the diathermy knife;

FIG. 2 is a drawing showing a puncture device and a sheath;

FIG. 3 is a cross-sectional view taken along line A-A in FIG. 2;

FIG. 4 is a drawing showing both two balloons inflated in the puncture device;

FIG. 5 is a drawing for illustrating procedures in which the endoscope is inserted into a stomach to effect puncture by using the puncture device;

FIG. 6 is a drawing showing a case where the puncture device is inserted from the abdominal region into the stomach and a process by which a fragile portion of a sheath is cut;

FIG. 7 is a drawing showing a case where the sheath is removed to retain only the puncture device;

FIG. 8 is a drawing showing a case where a target site on the stomach wall is approached by using as a guide an indicator on the inflated second balloon;

FIG. 9 is a drawing showing a case where an opening is formed on the stomach and a target site inside the abdominal cavity is approached by using as a guide an indicator on the inflated first balloon;

FIG. 10 is a drawing showing another aspect of an indicator on a holding portion; and

FIG. 11 is a drawing showing another aspect of the second balloon.

### DETAILED DESCRIPTION OF THE INVENTION

A detailed explanation will be made for embodiments by checking drawings.

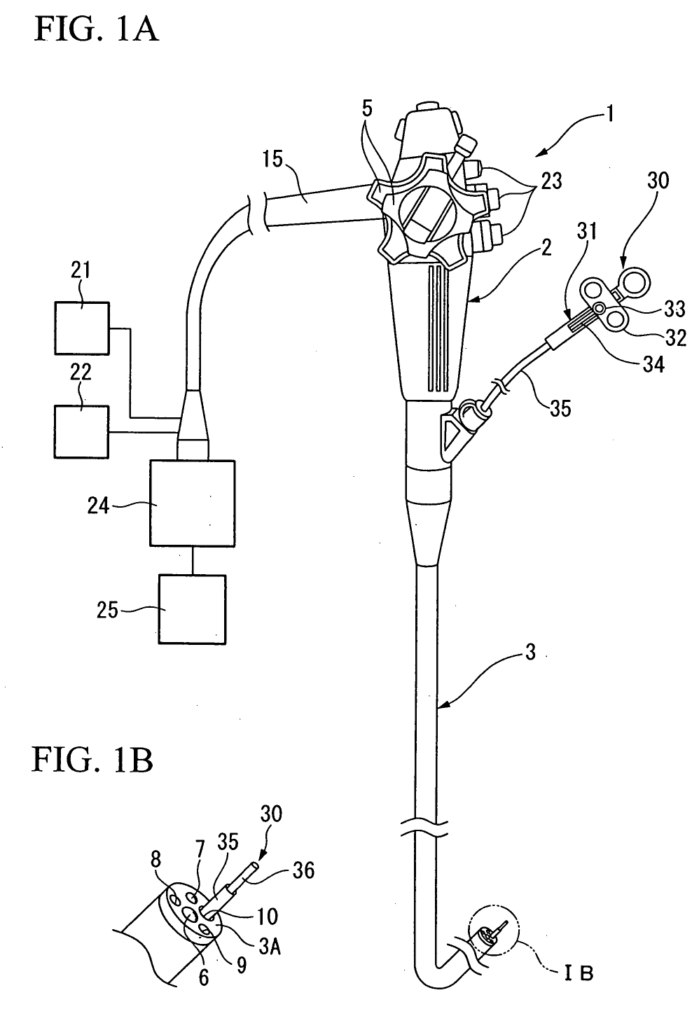

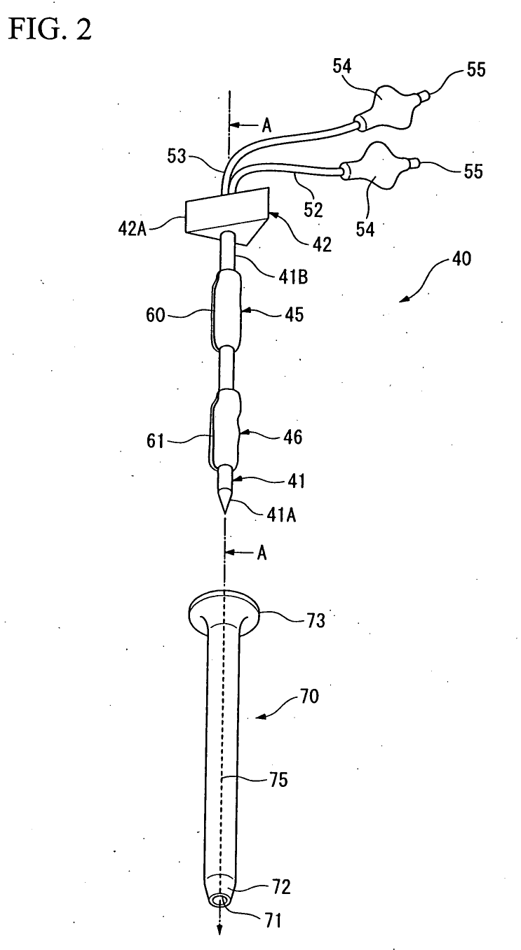

FIG. 1A and FIG. 1B show an endoscope as one example of the device. In an endoscope 1, an insertion portion 3 inserted into a living body of a patient is extended from an operational portion 2 used by an operator. The insertion portion 3 is elongated and flexible. The distal end 4 of the insertion portion 3 can be bent by using an angle knob 5 of the operational portion 2. As shown in FIG. 2, on the distal end surface 3A of the insertion portion 3 are arranged an observation device 6 for observing inside living body and a lighting device 7. The observation device 6 is constituted, for example, with an observation optical system such as an object lens and a CCD (Charged Coupled Device) as an imaging device. The lighting device 7 has a constitution in which illumination light is led by using an optical fiber from an external light source device. An output signal from the imaging device may be configured so as to be transmitted to a controller 24 to be described later through a signal wire which is passed into an insertion portion 3. Further, the output signal from the imaging device may be configured so as to be transmitted to the controller 24 by radio. Still further, the lighting device 7 may be constituted by using a known light emitting element (for example, light-emitting diode).

In addition, on the distal end surface 3A of the insertion portion 3 are arranged distal end openings of various channels 8 through 10. An air/fluid feeding channel 8 is a duct used in supplying fluid to living body. A suction channel 9 is a duct used in sucking fluid from living body. An operational channel 10 is a channel for allowing an treatment device to pass. Various channels 8 through 10 are extended from an insertion portion 3 to an operational portion 2. However, the constitution of the endoscope 1 is not restricted thereto. For example, it may be configured so that the suction channel 9 is omitted and the operational channel 10 is used to effect suction. Further, it may be provided with a plurality of the operational channels 10.

The base-end side opening of the operational channel 10 is provided at the side of the operational portion 2. Other channels 8 and 9 are connected via a universal cable 15 shown in FIG. 1 to an air/fluid feeding device 21 and a suction device 22.

The air and fluid feeding or the suction can be operated by using a button 23 arranged on the operational portion 2. Further, an endoscope 1 is connected via a universal cable to a controller 24 as well. The controller 24 is a device for controlling the endoscope 1 and equipped with an image processing device and a light source, providing a monitor 25 with various images (images).

FIG. 1 shows a diathermy knife 30, as an example of an treatment device which is inserted into an operational channel 10 of the endoscope 1. The diathermy knife 30 is attached to an operational portion 31 so that a slider 32 can freely advance and retreat. The slider 32 comprises a terminal 33 which is to be connected to a high-frequency electric source. The terminal 33 is connected to a coated electric-conductive wire 34. The electric-conductive wire 34 passes through a flexible sheath 35 extending from the distal end of the operational portion 31 and is connected to a treatment portion 36 at the distal end.

FIG. 2 shows a puncture device 40 inserted from the abdominal wall. The puncture device 40 comprises an elongated needle 41, and the distal end 41A of the needle 41 is edged so as to be inserted into living body. The proximal end 41B of the needle 41 comprises a holding portion 42 which is held by an operator. The holding portion 42 is formed in a triangle shape by view on arrow indicating the insertion direction. A first balloon (first cuff) 45 and a second balloon (second cuff) 46 are bound by a thread (not shown), etc., or welded on the outer periphery of the needle 41 sequentially from the proximal end side. The first and the second balloons 45 and 46 are attached at such a site that the first balloon 45 can be arranged inside the abdominal cavity when the second balloon 46 is inserted into a hollow organ, as will be described later. It is noted that the first and the second balloons 45 and 46 are manufactured with a flexible material safe for living body, for example, flexible vinyl chloride resin or silicone rubber.

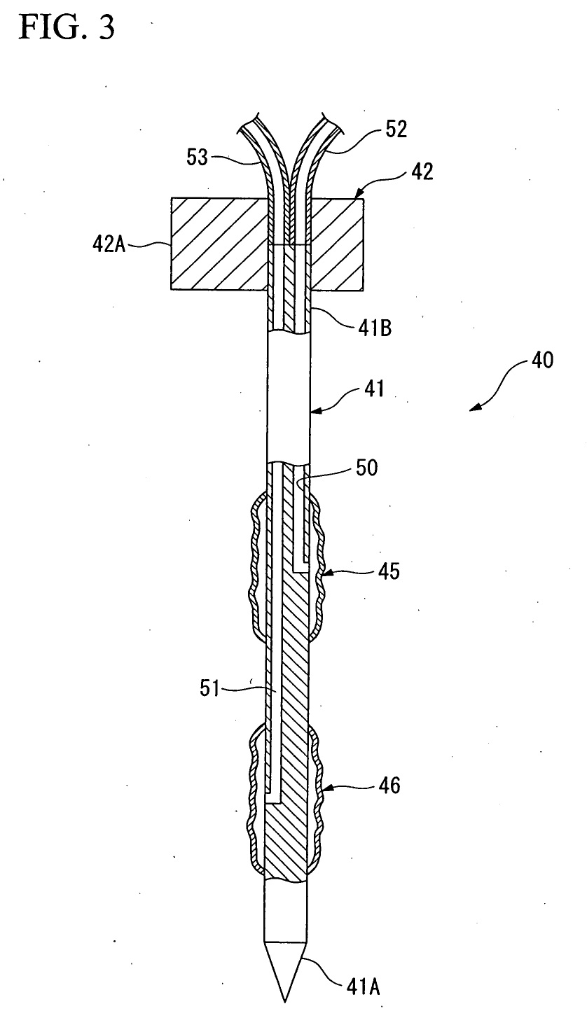

As shown in FIG. 3, the first balloon 45 is communicatively connected to the distal end opening of a first duct 50 formed inside a needle 41. The second balloon 46 is communicatively connected to the distal end opening of a second duct 51 formed inside the needle 41. The first duct 50 and the second duct 51 are independent ducts and respectively connected to other tubes 52 and 53. As shown in FIG. 2, at the proximal ends of these tubes 52 and 53 are provided a seal portion 54 such as a check valve and subsequently a connector 55, to which one fluid feeder such as a syringe can be connected respectively.

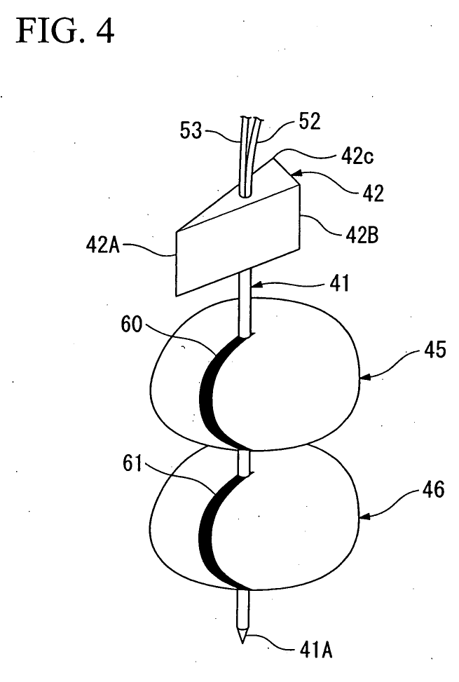

The first and the second balloons 45 and 46 are deflated along the outer periphery of a needle 41 in an initial state and the outer diameter of a puncture device 40 is sized so as to be slightly larger than the outer diameter of the needle 41. In contrast, as shown in FIG. 4, upon supply of a fluid from the respective tubes 52 and 53, the first and the second balloons 45 and 46 are allowed to be inflated in a radial direction. Since the tubes 52 and 53 as well as the ducts 50 and 51 are independent, the first and the second balloons 45 and 46 are allowed to be independently inflated or deflated. In this instance, an indicator (abdominal cavity indicator) 60 colored separately in a line shape is provided on the outer surface of the first balloon 45. An indicator (hollow organ indicator, a first indicator) 61 colored separately in a line shape is provided at the same site with the indicator 60 of the first balloon 45 on the outer surface of the second balloon 46. Further, a corner 42A of a holding portion 42 is arranged so as to be made aligned with the direction at which the indicators 60 and 61 are faced, as viewed on arrow in the insertion direction. The corner 42A is lower in angle than other two corners 42B and 42C, and an indicator (a second indicator) which provides an easier visual recognition.

As shown in FIG. 2, it is preferable that the puncture device 40 is inserted into the abdominal wall, while inserted into an insertion-assisting sheath 70. The sheath 70 is a hollow member in which a needle 41 and the respective balloons 45 and 46 can be inserted and shorter in length than the needle 41. The distal end 41A of the needle 41 is allowed to protrude from a distal end opening 71, and a tapered surface 72 radially expanding toward the proximal end is provided in the vicinity of the distal end opening 71. A base-end 73 is expanded radially like a flange so as to be easily held by an operator. Further, the sheath 70 comprises a fragile portion 75 from the distal end opening 71 to the outer periphery surface of the proximal end 73 in a length direction. FIG. 2 shows one example of the fragile portion 75 in which cuts are given at an equal space in the length direction. The fragile portion may be decreased in thickness at an equal space over the whole length. Such a sheath 70 is manufactured with a flexible material safe for living body, for example, flexible vinyl chloride resin or silicone rubber. Further, a puncture device may be constituted with the puncture device 40 exemplified in the embodiment including the sheath 70.

Next, an explanation will be made for actions of the embodiment. Hereinafter, an explanation will be made for procedures in which an endoscope 1 inserted, for example, from the mouth of a patient is used to form an opening in the stomach wall, as a natural orifice of living body, and approached into the abdominal cavity to perform preferred a medical procedure to organs and tissues (hereinafter, referred to as target organs) inside the abdominal cavity. The natural orifice into which the endoscope 1 is inserted is not restricted to the mouth but may be formed in the nose and the anus. Further, treatments as a medical procedure include various actions such as suture, observation, incision, collection of cells and removal of an organ.

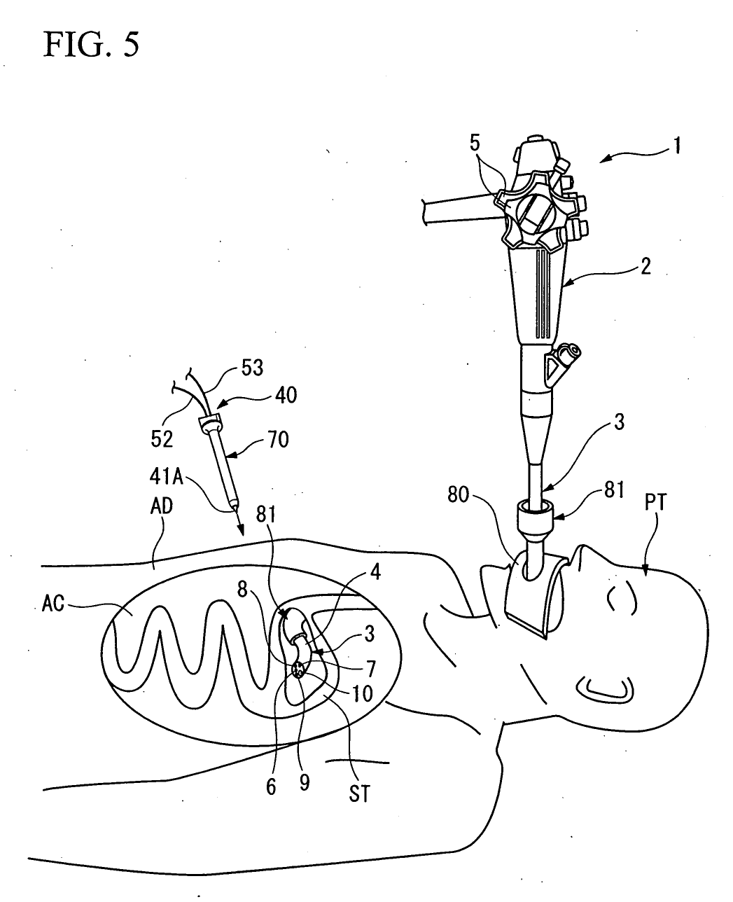

As shown in FIG. 5, a patient is placed so that his/her abdominal region AD faces above and an endoscope 1 is inserted from the mouth of the patient. It is preferable to use a mouth piece 80 and an over tube 81 in inserting the endoscope 1. The over tube 81 is used as a guide tube in a device having an insertion portion such as the endoscope 1, when the device is inserted into or removed from living body. However, the device may be inserted into living body, without use of the over tube.

After the endoscope 1 is inserted into the stomach ST, air is supplied from an air/fluid feeding channel 8 (duct) of the endoscope 1 to inflate the stomach ST, thereby, securing the field of vision and a space into which a puncture device 40 is inserted. Further, a space is decreased between the stomach wall and the abdominal wall. Next, disinfection is performed mainly to a site near the outside of the stomach on living body (the surface of the abdominal region at which the stomach is located in the embodiment), and the puncture device 40 is inserted toward the stomach ST from the abdominal region AD, with the sheath 70 covered on the puncture device 40. The puncture device 40 is predetermined for a length in a state that when a holding portion 42 is pressed to an extent that it is substantially in contact with the outer surface of the abdominal region AD, the first balloon 45 is arranged at the abdominal cavity AC and the second balloon 46 is arranged inside the stomach ST.

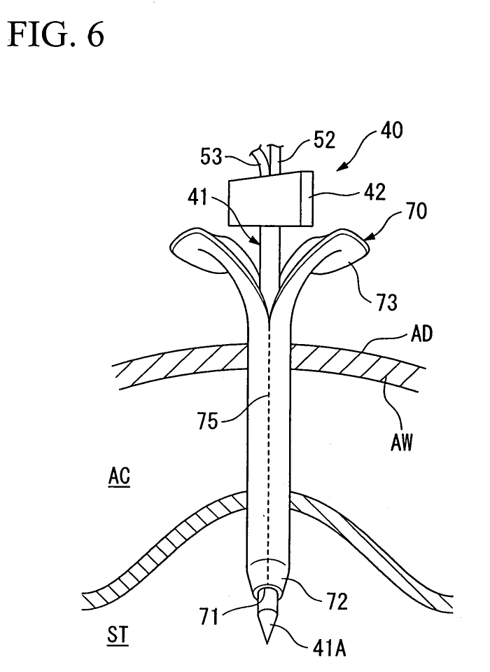

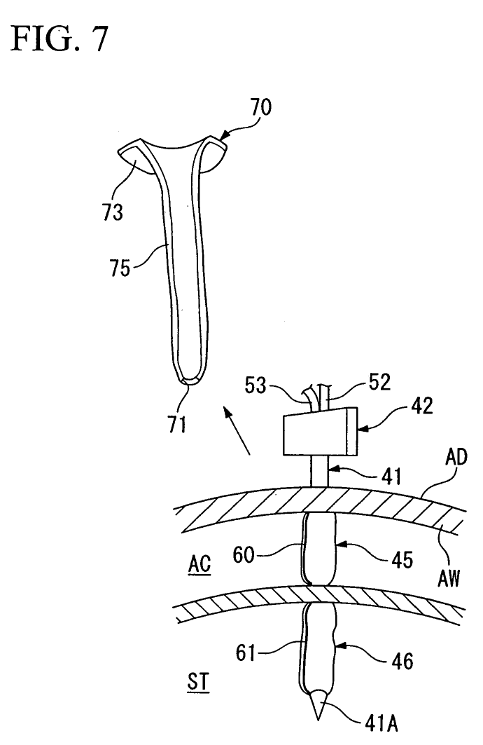

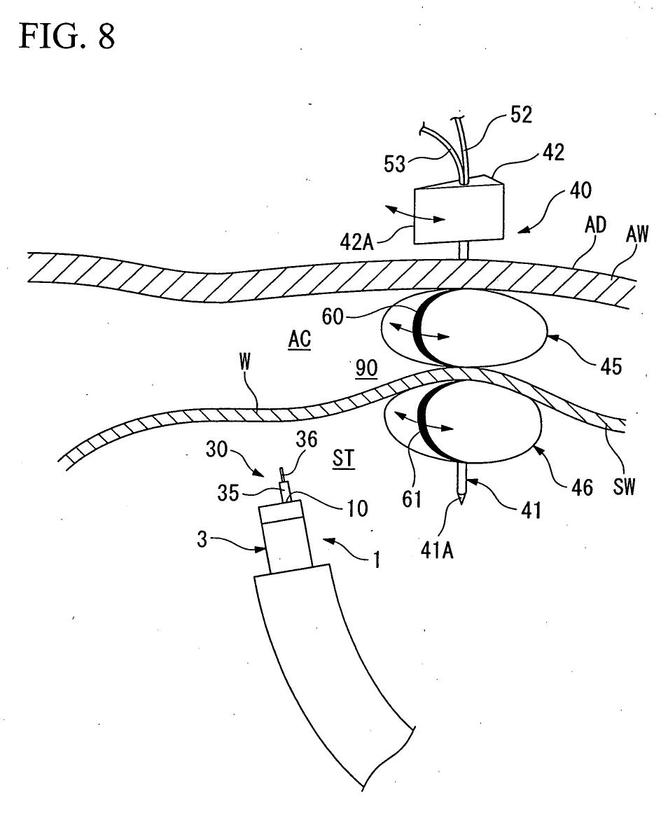

After the puncture device 40 is inserted, as shown in FIG. 6, an operator breaks a fragile portion 75, holding the flange-shaped proximal end 73 of the sheath 70. As shown in FIG. 7, the sheath 70 is broken and taken outside living body, thereby retaining the needle 41 in living body. A syringe containing a fluid such as physiological saline solution or CO₂ is loaded respectively in the tubes 52 and 53, thereby inflating the respective balloons 45 and 46. As shown in FIG. 8, the first balloon 45 is inflated so as to provide a desired space between the abdominal wall AW and the anterior wall SW inside the abdominal cavity AC, and a space 90 according to the dimension of the first balloon 45 inside the abdominal cavity AC is provided. Further, the second balloon 46 is inflated inside the stomach ST. The second balloon 46 prevents the puncture device 40 from being pulled out from the stomach ST and also indicates a predetermined direction, using an indicator 61.

At this timing, an operator may hold a holding portion 42 and turns it in the direction as shown view on arrow, thereby making it possible to set a target site W to a desired direction. For example, when the corner 42A of the holding portion 42 is indicated so that the target site W of the anterior wall SW, as a site at which an opening is to be formed, is allowed to align with the direction of a target organ, the second balloon 46 inside the stomach ST rotates together, and the indicator 61 is directed toward the target site W. An operator confirms the direction of an indicator 61 by checking an observation device 6 of the endoscope 1, and faces the distal end of the endoscope 1 toward the direction.

Next, a diathermy knife 30 is used to incise the anterior wall SW in order to form an opening. In this instance, where the target site W is close to a site at which the puncture device 40 is inserted, the second balloon 46 may be deflated, after confirmation of the direction by using as a guide an indicator 61 of the second balloon 46. Since the second balloon 46 and the first balloon 45 are respectively connected to independent ducts 50 and 51, the first balloon 45 can be kept inflated. In this instance, the first balloon 45 is caught on the abdominal wall AW, thereby preventing the puncture device 40 from being removed outside living body.

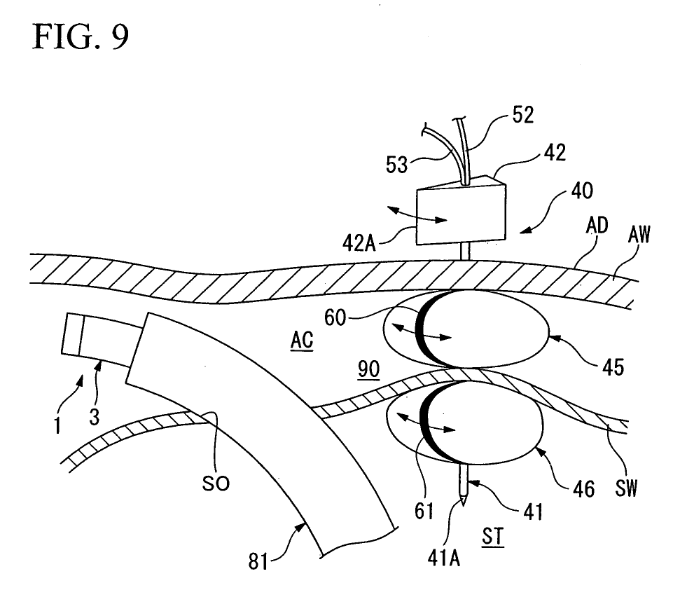

As shown in FIG. 9, after an opening SO is formed at a target site W of the anterior wall SW, the endoscope 1 and the over tube 81 are introduced via the opening SO into the abdominal cavity AC. Since the first balloon 45 keeps the distance between the anterior wall SW and the abdominal wall AW, the action of securing a space between the stomach wall and the abdominal wall can be omitted in forming the opening SO.

Further, in this instance, an insufflation needle may be inserted into the abdominal region to feed carbon dioxide into the abdominal cavity, in order to attain insufflation. It is desirable to inflate the abdominal cavity AC for securing a space for performing A medical procedure in the abdominal cavity AC. However, if a desired space can be secured, insufflation may not be necessarily attained. Further, a method for inflating the abdominal cavity AC is not restricted to a method of inflation with a gas but any known abdominal lifting method may be used for securing a space inside the abdominal cavity. In addition, the abdominal cavity AC may be insufflated via a channel of the endoscope 1.

A direction of allowing the endoscope 1 to advance inside the abdominal cavity AC can be confirmed by using as a guide an indicator 60 of the first balloon 45. An operator faces the corner 42A of the holding portion 42 toward a target site inside the abdominal cavity AC. As a result, since the indicator 60 of the first balloon 45 indicates the direction of the target site inside the abdominal cavity AC, an operator advances the endoscope 1, while confirming the position of the indicator 60 by checking an image of the endoscope 1. An approximate direction of a target organ with respect to a hollow organ (the stomach ST in the present embodiment) where an opening is formed can be grasped outside living body. Therefore, in confirmation of a target site where an opening is formed, an approximate direction at which the target organ is located is determined from outside living body to adjust the indicator for direction from outside living body so as to indicate the direction. Then, the direction indicated by the indicator 61 (or indicator 60) is confirmed by checking an image of the endoscope 1, thereby making it possible to recognize an approximate direction of the target site with guidance to the image of the endoscope 1.

After confirmation of a target site inside the abdominal cavity AC by using an observation device of the endoscope 1, treatments are performed via necessary treatment devices through an operational channel 10. After completion of the treatments inside the abdominal cavity AC, an endoscope 1 and an over tube 81 are pulled back into the stomach ST and a suture instrument, etc., are used to seal an opening SO. The puncture device 40 is pulled out after a fluid supplied to the puncture device 40 is discharged and the first and the second balloons 45 and 46 are deflated. Then, the endoscope 1 and the over tube 81 are pulled outside living body.

Further, a puncture device 40 can be used not only in a case where the endoscope 1 is introduced into the abdominal cavity AC but also in a case where A medical procedure are performed in the stomach ST. For example, when mucous membrane is excised from the stomach ST, the puncture device 40 is inserted near a target site. As explained previously, the first balloon 45 is inflated to form a space between the anterior wall SW and the abdominal wall AW in the stomach ST, and the second balloon 46 is also inflated to indicate the position inside the stomach ST. A injection needle is inserted through an operational channel 10 of the endoscope 1 and inserted to an extent that the distal end of the injection needle reaches a space between the mucus membrane of the anterior wall SW and the muscular layer. Further, physiological saline solution is supplied from a syringe attached to the proximal end of the injection needle to swell the mucous membrane. The injection needle is pulled out from the operational channel 10, and resection forceps, a diathermy knife, a snare, etc., are then inserted through the operational channel 10 to excise the swollen mucous membrane. Since the space 90 is secured by the first balloon 45 between the anterior wall SW and the abdominal wall AW, the abdominal wall AW or other organs are not damaged in the treatment of the anterior wall SW.

According to the present embodiment, the puncture device 40 is inserted into the anterior wall SW of the stomach ST from the abdominal wall AW, and the distal end thereof is confirmed by using an observation device 6 of the endoscope, which facilitates confirmation of the location of the anterior wall SW in the stomach ST. Further, inflation of the second balloon 46 inside the stomach ST, which facilitates confirmation of the location in the stomach ST by checking the observation device 6 of the endoscope 1. Since the puncture device 40 is inserted from the abdominal wall AW side, confirmation can be easily made for where the anterior wall SW in the stomach ST is located. The endoscope 1 can be introduced into the abdominal cavity AC via the stomach ST away from the omentum or other organs, thereby making it possible to easily perform procedures.

Since the indicator 61 is provided at the second balloon 46 of the puncture device 40, it is allowed to be used as a guidance in operating the endoscope 1 inside the stomach ST. In a conventional method, conventionally, because it is difficult to confirm upper, lower, left, and right positions, or a site to be treated inside the stomach ST only with an image of the endoscope, experience in treating a patient is required. In the present embodiment, the site can be easily confirmed to reduce the burden on an operator. Further, since the direction of the indicator 61 of the second balloon 46 is made aligned with that of the indicator outside living body (corner 42A), the endoscope 1 can be guided toward the direction intended by an operator. As a result, procedures can be performed easily to reduce the burden on an operator or a patient.

A space 90 can be formed between the anterior wall SW and the abdominal wall AW by providing a first balloon 45. Therefore, the presence of the abdominal wall AW may be neglected in performing procedures for the anterior wall SW. Further, when the endoscope 1 is allowed to advance into the abdominal cavity AC via the stomach ST, the space can be used to easily advance the endoscope 1 thereinto. In addition, since the indicator 60 is provided on the first balloon 45, it is possible to easily grasp the location inside the abdominal cavity AC, which is otherwise difficult by a conventional method.

Since the first and the second balloons 45 and 46 are allowed to be inflated or deflated independently, treatments can be performed even at a site close to the puncture device 40. Inflation of at least either of the balloon 45 or 46 is reliably able to prevent the puncture device 40 from being pulled out, if the other balloon 45 or 46 is deflated.

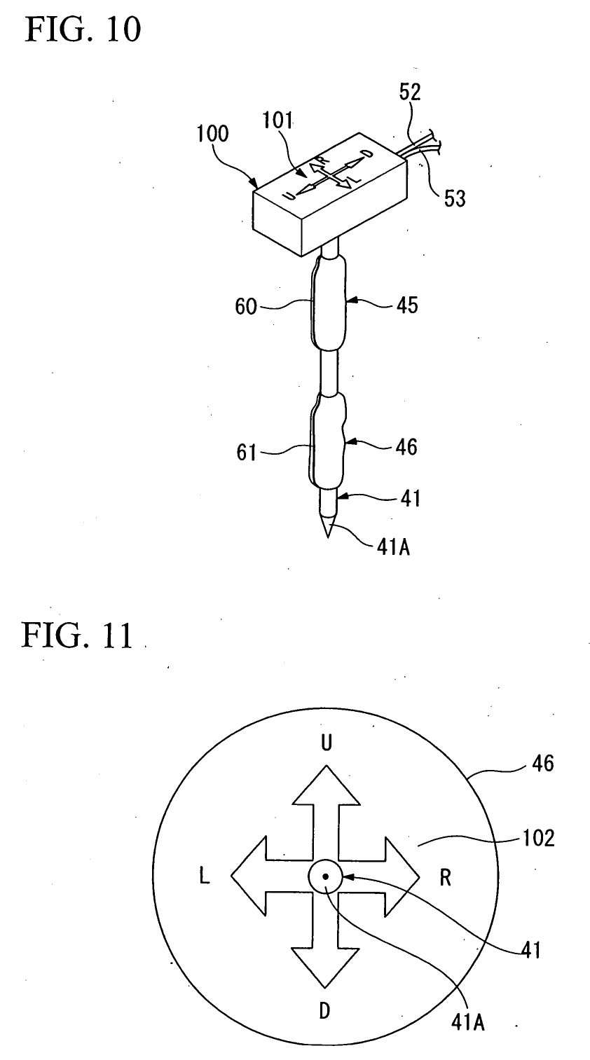

The indicators 60 and 61 which function as a direction indicator may be attached to at least either of the first balloon 45 or the second balloon 46. The indicators are not restricted in aspect only to a color separation in a line shape but may be available in a circular shape or a rectangular shape. An indicator on the holding portion 42 side is not restricted to a triangular shape but may be available in another shape, for example, a pentagonal shape. The indicator may also be provided on the holding portion 42 as projections and depressions or in separate colors. Further, the indicator 101 of the holding portion 100 shown in FIG. 10 and the indicator 102 of the second balloon 46 shown in FIG. 11 may be used as well. FIG. 10 is a drawing of the puncture device observed from above and the indicator 101 is provided on the upper surface of the holding portion 100, and made up of the arrow and letters arranged up, down, left, and right. FIG. 11 is a drawing of the second balloon 46 in an inflated state observed from below. The indicator 102 is made up of the arrow and letters arranged up, down, left, and right. Every direction of the indicator 102 aligns with that of the indicator 101 in FIG. 10. Thus constituted indicators 101 and 102 also provide the same effect.

The present invention is not restricted to the above embodiment, despite the foregoing explanation about the embodiment. In the present invention, the constitution may be added, omitted, replaced or exchanged in any way, within a scope not deviated from the object of the invention. The present invention is not restricted to the above explanation but restricted only by the claims attached herewith.

For example, a hollow organ in which an opening SO is formed is not restricted only to the stomach ST. The hollow organ may include, for example, the esophagus, duodenum, small intestines, large intestines, uterus and bladder.

A device necessary for performing desired procedures is not restricted to an endoscope equipped with the observation device and the operational channel described in the above embodiments. For example, such a device may be used that comprises a treatment portion for performing desired procedures at the distal end side of an insertion portion inserted into living body and an operational portion capable of operating the treatment portion outside living body. Further, where an treatment device is not provided with an observation device, various modes are available, for example, a aspect in which the above-described capsule endoscope is used together. In addition, as another example of the above-described treatment device, a device may be used, which has a lumen into which an treatment device can be inserted at an insertion portion but not provided with an observation mechanism.

What is claimed is:

1. A medical procedure performed via a natural orifice comprising:
inserting a puncture device from an abdominal wall side into the vicinity of a target site in a hollow organ, and protruding the distal end thereof inside the hollow organ;
introducing an observation device into the hollow organ via a natural orifice of a living body, and setting a target site at the hollow organ by checking the puncture device as an indicator; and
treating the target site confirmed by the observation device by using an treatment device inserted via a natural orifice.

2. The medical procedure performed via a natural orifice as set forth in claim 1, wherein the inserting the puncture device comprises:
placing a balloon provided on the puncture device between the abdominal wall and the hollow organ; and
inflating the balloon after inserting the puncture device.

3. The medical procedure performed via a natural orifice as set forth in claim 1, wherein the inserting the puncture device comprises:
placing a first balloon provided at the puncture device between the abdominal wall and the hollow organ; and
placing a second balloon inside the hollow organ, the second balloon being attached to a site closer to the distal end side of the puncture device than a site to which the first balloon is attached.

4. The medical procedure performed via a natural orifice as set forth in claim 3, wherein the confirming a target site comprises using an indicator provided to the second balloon in order to confirm the direction inside the hollow organ.

5. The medical procedure performed via a natural orifice as set forth in claim 3, wherein the confirming a target site comprises inserting the observation device, provided at the distal end of an insertion portion which is flexible and inserted via the natural orifice into the hollow organ, and
the treatment inside the hollow organ comprises inserting the treatment device into the hollow organ via a duct provided to the insertion portion.

6. The medical procedure performed via a natural orifice as set forth in claim 1, comprising supplying air into the hollow organ for inflation prior to the protrusion of the distal end of the puncture device inside a hollow organ.

7. The medical procedure performed via a natural orifice as set forth in claim 6, wherein the treating the target site comprises forming an opening on the wall of the hollow organ.

8. The medical procedure performed via a natural orifice as set forth in claim 7, further comprising introducing a device which is introduced via the opening into the abdominal cavity to perform a second treatment inside the abdominal cavity.

9. A puncture device comprising:
a needle configured to be inserted from an abdominal wall into a hollow organ;
a first balloon attached to the needle and inflatable by supply of a fluid;
a second balloon attached to a site closer to the distal end side of the needle than a site to which the first balloon is attached and inflatable by supply of the fluid; and
a duct which supplies the fluid respectively to the first balloon and the second balloon.

10. The puncture device as set forth in claim 9, wherein the second balloon comprises an indicator configured to indicate the direction inside the hollow organ so as to be observable on inflation.

11. The puncture device as set forth in claim 9, comprising
a first indicator provided to the second balloon and observable for the direction inside the hollow organ on inflation; and
a second indicator provided at the proximal end of the needle which stays outside a living body and indicates a direction with respect to the direction indicated by the indicator of the second balloon.

12. The puncture device as set forth in claim 9, wherein the duct is capable of inflating independently the first balloon and the second balloon.

13. The puncture device as set forth in claim 9, comprising a sheath covering the respective outer peripheries of the first balloon and the second balloon when the first balloon and the second balloon are deflated.

14. The puncture device as set forth in claim 13, wherein the sheath comprises a fragile portion which makes the sheath removable by partially breaking the sheath after insertion into the living body.
